(19) United States
(12) Patent Application Publication
(10) Pub. No.: US 2003/0199962 A1
(43) Pub. Date: Oct. 23, 2003

(54) Anti-slip leads for placement within tissue

(76) Inventors: Chester Struble, Eijsden (NL); Pierre Grandjean, Warsage (BE); Karel F.A.A. Smits, Munslergsleen (NL)

Correspondence Address: MEDTRONIC, INC., 710 MEDTRONIC PARKWAY NE, MS-LC340, MINNEAPOLIS, MN, 55432-5604, US

(21) Appl. No.: 10/127,041

(22) Filed: Apr. 22, 2002

(51) Int. Cl. A61N 1/05

(52) U.S. Cl. 607/126

(57) ABSTRACT

In one embodiment, the invention is directed to an implantable medical lead comprising a lead including a proximal end, a distal end, and an electrode. The lead may also include tissue fixation structures on the lead a distance from the electrode, wherein the tissue fixation structures exploit acute and chronic fibrous tissue growth with respect to the lead to enhance fixation of the lead to tissue and thereby secure the electrode in an implanted position. In some cases, the tissue fixation structures may include fibrous growth holes to exploit acute and chronic fibrous tissue growth and advantageously anchor the lead within tissue.

### FIELD OF THE INVENTION

The invention relates to implantable medical devices, and more particularly, to implantable leads of medical devices.

### BACKGROUND

In the medical field, leads are used with a wide variety of medical devices. For example, leads are commonly implemented to form part of implantable cardiac pacemakers that provide therapeutic stimulation to the heart by delivering pacing, cardioversion or defibrillation pulses. The pulses can be delivered to the heart via electrodes disposed on distal ends of the leads. In that case, the leads may position the electrodes with respect to various cardiac locations so that the pacemaker can deliver pulses to the appropriate locations. Leads are also used for sensing purposes, or both sensing and stimulation purposes.

Leads are also used in neurological devices such as deep-brain stimulation devices, and spinal cord stimulation devices. For example, the leads may be stereotactically probed into the brain to position electrodes for deep brain stimulation. Leads may also be used with a wide variety of other medical devices including, for example, devices that provide muscular stimulation therapy, and the like. In each case, the leads may be used for sensing purposes, stimulation purposes, or both.

In many cases, electrodes need to be precisely positioned within cellular tissue. Accordingly, fixation of the lead with respect to the target tissue site is of paramount concern. Lead dislocation or migration can result in inoperability of the medical therapy, and possibly the need for additional medical procedures to re-position the lead. Conventional lead designs have employed a variety of structures to achieve fixation with respect to organ tissue or other cellular tissue. For example, conventional leads have implemented coiled screw configurations, fish-hook configurations, flanges or tines. Many of these conventional leads, however, provide relatively aggressive fixation techniques, making removal of the leads quite difficult, should removal become necessary. In particular, leads implemented with conventional fixation techniques may cause substantial tissue mutilation upon removal. Table 1 below lists a number of documents that disclose implantable devices that use various conventional implantable leads.

TABLE 1

| Patent No. | Inventor | Issue Date |
|---|---|---|
| 5,728,140 | Salo et al. | Mar. 17, 1998 |
| 5,683,446 | Gates | Nov. 4, 1997 |
| 5,374,287 | Rubin | Dec. 20, 1994 |
| 3,857,399 | Zacouto | Dec. 31, 1974 |
| 6,263,250 | Skinner | Jul. 17, 2001 |
| 6,078,840 | Stokes | Jun. 20, 2000 |
| 6,049,736 | Stewart et al | Apr. 11, 2000 |

All patents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, the devices and methods disclosed in the patents of Table 1 may be modified advantageously by using the techniques of the present invention.

### SUMMARY OF THE INVENTION

The present invention has certain objects. That is, various embodiments of the present invention provide solutions to one or more problems existing in the prior art with respect to medical devices in general, and fixation of medical leads in particular. These problems include, for example, a failure to achieve adequate fixation of leads within cellular tissue. Also, conventional leads may fail to achieve removable fixation to tissue without causing substantial tissue mutilation upon removal of the lead. Various embodiments of the present invention have the object of solving at least one of the foregoing problems.

It is an object of the invention to improve implantable medical leads. It is a further object of the invention to improve fixation of implantable leads to cellular tissue so that an electrode disposed on a distal end of the lead can be positioned with respect to a desired location. Accordingly, it is another object to improve the precision of electrode positioning within tissue. For example, the invention may be particularly useful in fixing leads within the interventricular septum such that an electrode disposed on a distal end of the lead is positioned in close proximity to the left ventricle.

It is a further object of the invention to provide tissue fixation structures near a distal end of an electrode. A number of different embodiments of various tissue fixation structures are outlined below. Tissue fixation structures can facilitate fixation of medical leads and avoid both acute and chronic lead dislocation or migration. In particular, tissue fixation structures can exploit and harness both normal and fibrous tissue growth within the tissue to anchor the lead in a fixed location. It is a further object of the invention to exploit fibrous tissue growth to advantageously anchor the lead within tissue.

The invention may offer one or more advantages. For example, the invention may improve both acute and chronic fixation of a lead within tissue. In this manner, more precise placement of electrodes within tissue can be achieved. Moreover, the invention may include features to more filly exploit tissue growth to enhance the fixation effect. Still, the invention may allow fixation of leads in a manner that does not cause substantial tissue mutilation upon removal of the lead from the tissue.

Various embodiments of the invention may possess one or more features capable of fulfilling the above objects. In general, the invention provides an implantable medical lead comprising a lead including a proximal end, a distal end, and one or more electrodes. The lead may also include tissue fixation structures formed on the lead at a distance from the electrode. The tissue fixation structures exploit fibrous tissue growth with respect to the lead to enhance fixation of the lead to tissue and thereby secure the electrode in an implanted position. In some cases, the tissue fixation structures may include fibrous growth structures, such as holes, to exploit tissue encapsulation and fibrous tissue growth and advantageously anchor the lead within tissue.

In another embodiment, the invention is directed to an implantable medial device, such as a cardiac pacemaker, a neurological stimulating device, or a muscular stimulating device. In any case, the implantable medial device comprises at least one lead that includes tissue fixation structures that improve tissue encapsulation and fibrous tissue growth with respect to the lead to enhance fixation of the lead to tissue and thereby secure the electrode in an implanted position. The implantable medical device may also include a control unit coupled to the lead to provide signal processing of signals detected by the lead, or to deliver therapeutic stimuli to the patient via the lead.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a simplified schematic view of an implantable medical device with an enlarged view of a distal end of an implantable lead.

FIG. 2 is a perspective view of a lead implanted within a patient as a deep brain stimulation lead.

FIG. 3 is a cross-sectional view of a heart having a lead implanted within the interventricular septum.

FIG. 4 is a block diagram illustrating the constituent components of an implantable medical device in the form of an exemplary cardiac pacemaker useful with a lead as shown in FIG. 3.

FIGS. 5-9 are close-up cross-sectional side-views of various embodiments of a distal end of a lead in accordance with the invention.

FIG. 10 is an enlarged front-view of the lead illustrated in FIG. 9, showing fibrous growth holes formed in one of several ring structures.

FIGS. 11A-11C are enlarged cross-sectional side-views collectively illustrating the insertion and placement of a lead within tissue.

FIG. 12 is another enlarged cross-sectional side-view in which a lead that includes distal tines is inserted and placed within tissue.

FIG. 13 is an enlarged cross-sectional side-view of an embodiment of a distal end of a lead in accordance with the invention.

FIG. 14 is an enlarged front-view of the lead illustrated in FIG. 13, showing non-continuous spiral ring structures including fibrous growth holes formed in the non-continuous spiral ring structures.

FIG. 15 is an enlarged cross-sectional side-view illustrating the insertion and placement of the lead illustrated in FIGS. 13 and 14 within tissue.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is to be understood that other embodiments may be utilized and structural or logical changes may be made without departing from the scope of the present invention. The following detailed description, therefore, is not to be taken in a limiting sense, and the scope of the present invention is defined by the appended claims.

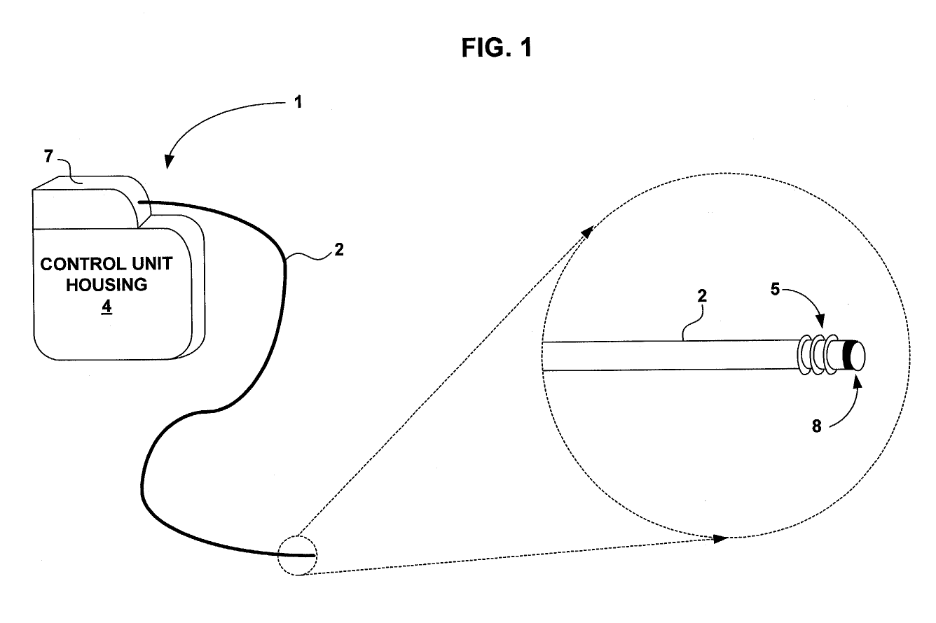

FIG. 1 is a simplified schematic views of an implantable medical device 1 that includes an implantable lead 2. FIG. 1 also provides a close up view of the distal end of lead 2. As outlined in detail below, implantable lead 2 incorporates tissue fixation structures 5 to facilitate fixation with respect to organ tissue or other cellular tissue. Implantable lead 2 may include one or more electrodes, such as electrode 8 positioned at the distal end of implantable lead 2. Thus, fixation of structures 5 with respect to organ tissue can likewise fixedly position electrode 8 with respect to a desired location within a patient. Signals can then be sensed or stimulation pulses can be delivered to the patient via electrode 8. Implantable lead 2 can be secured within the tissue by tissue growth through and around the tissue fixation structures 5, and may be removable from the tissue without causing substantial tissue mutilation.

Lead 2 has a proximal end (connected to a control unit within control unit housing 4). For example, the proximal end may be connected to a connector module 7, which in turn is coupled to sensing circuitry and/or stimulation circuitry of the control unit within control unit housing 4. Lead 2 may be formed from a biocompatible material such as silicone rubber, polyurethane, a silicone-polyurethane copolymer with or without surface modifying end groups. An electrode 8 may be positioned at the distal end of lead 2. In addition, any number of additional electrodes (not shown) may be distributed along the length of lead 2.

Electrode 8, as well as other electrodes (if desired) can be made from an electrically conductive, biocompatible material such as elgiloy, platinum, platinum-iridium, platinum-iridium oxide, sintered platinum powder or other residue product after combustion with some high heat source, platinum coated with titanium-nitride, pyrolytic carbon (made via some pyrotechnics manufacturing technique), or the like. In addition, one or more of electrodes 8 may function as sensing electrodes to monitor internal electrical signals of the human patient or other mammal in which device 1 is implanted. Although a single lead 2 is shown for purposes of illustration, any number of leads may be used, and thus coupled to connector module 7. In some embodiments, a reference potential may be provided not by the electrode carried on lead 2, but by an external reference electrode or a contact surface on an implanted pulse generator or the like.

Electrode 8 may form a substantially cylindrical ring of conductive material that extends about an exterior wall of lead 2. For example, an electrode 8 may extend the entire 360 degrees about lead 2 or some lesser extent. In some embodiments, lead 2 may be tubular but not necessarily cylindrical. For example, electrode 8 and lead 2 may have alternative cross sections, e.g., square, rectangular, hexagonal, oval or the like. Electrode 8 may be coupled to an internal conductor that extends along the length of lead 2.

Implantable medical device 1 may comprise any device that incorporates one or more implantable leads. For example, implantable medical device 1 may take the form of an implantable brain stimulation device, an implantable muscular stimulation device, a cardiac pacemaker, cardioverter or defibrillator, or the like. In some embodiments, control unit housing 4 may be external (not implanted), with lead 2 forming an implantable portion of the device. In other embodiments, control unit housing 4 and lead 2 are both implanted within a patient.

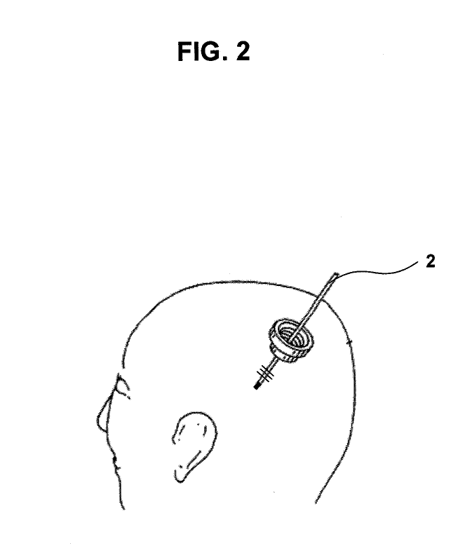

FIG. 2 is a perspective view of lead 2 implanted within a patient as a deep brain stimulation lead. In the example of FIG. 2, control unit housing 4 (FIG. 1) may house circuitry that stimulates the brain according to deep brain stimulation techniques known in the art. Lead 2 illustrated in FIG. 2 may include various tissue fixation structures outlined in greater detail below to improve fixation to of lead 2 to brain tissue.

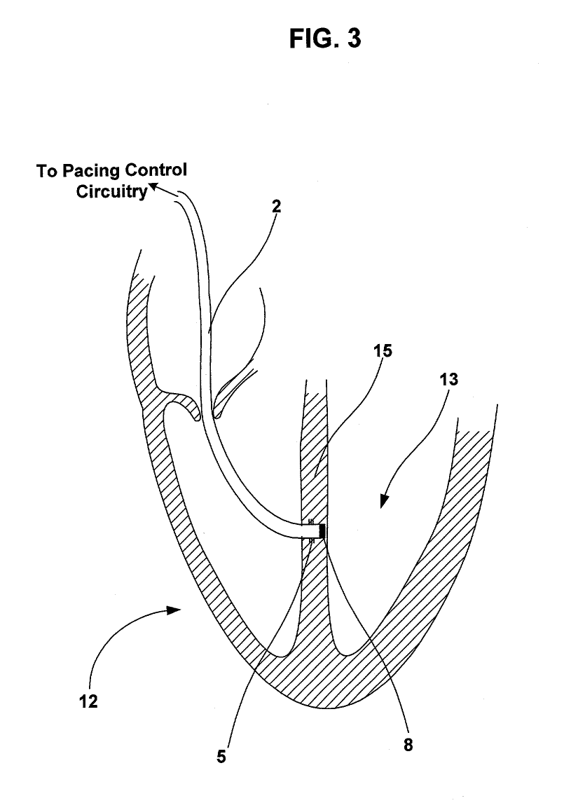

FIG. 3 is a cross sectional view of a heart 12 having lead 2 implanted within the interventricular septum 15. In the example of FIG. 3, control unit housing 4 (FIG. 1) may comprise circuitry that performs various cardiac sensing and pacing functions. Various details of an exemplary embodiment of implantable medical device 1 are provided below specifically for an implantable medical device in the form an implantable cardiac pacemaker. The invention, however, is not necessarily limited to use with cardiac pacemakers, but can be readily implemented as part of any of a wide variety of implantable medical devices.

In the cardiac pacemaker example, the pacemaker may comprise any number of pacing and sensing leads 2 (one lead shown) attached to a connector module of a hermetically sealed housing (like that shown in FIG. 1) and implanted within a human or mammalian patient. In that case, the pacing and sensing leads 2 sense electrical signals attendant to the depolarization and repolarization of the heart 12, and further provide pacing pulses for causing depolarization of cardiac tissue in the vicinity of the distal ends thereof. Pacing and sensing lead 2 may have unipolar or bipolar electrodes disposed thereon, as is well known in the art. Examples of a pacemaker include implantable cardiac pacemakers disclosed in U.S. Pat. No. 5,158,078 to Bennett et al., U.S. Pat. No. 5,312,453 to Shelton et al., or U.S. Pat. No. 5,144,949 to Olson, all hereby incorporated by reference herein, each in its respective entirety.

In particular, pace/sense electrode 8 senses electrical signals attendant to the depolarization and repolarization of the left ventricle 13 heart 12. The electrical signals are conducted to a pacemaker control unit within the hermetically sealed housing via lead 2. Pace/sense electrode 8 may also deliver pacing pulses for causing depolarization of cardiac tissue in the vicinity of left ventricle 13. The pacing pulses are generated by the pacemaker and are transmitted to pace/sense electrode 8 via lead 2.

A number of embodiments of lead 2 are outlined in greater detail below. In particular, lead 2 includes tissue fixation structures 5 that improve fibrous cell growth around lead 2, to thereby secure lead 2 in a fixed position within interventricular septum 15. Thus, tissue fixation structures 5 facilitate the ability to fixedly position electrode 8 in close proximity to left ventricle 13. For example, lead 2 can be secured within interventricular septum 15 by cell tissue growth around tissue fixation structures 5, and may be later removed from interventricular septum 15 without causing substantial tissue mutilation to interventricular septum 15. In particular, the removal of the lead may still cause some tissue mutilation, but the mutilation caused should be significantly less than mutilation caused by the removal of more aggressive designs such as screw-in lead or fish hook lead designs.

Typically a lead is removed by one of a variety of different methods. In one example, constant steady pulling on lead over a period of time (minutes, hours, or even a day or more) with a weight or a pulley attached to the proximal part of the lead until the lead tip lets loose. Another method to remove a lead involves placing a locking stylet in the internal lumen of the lead and advancing the locking stylet as deep as possible, preferably bottoming against the distal electrode tip. Then the locking mechanism can be activated whereby the locking stylet is locked against the electrode tip or distal most portion of the conductor coil. The locking stylet can then be pulled such that the pull force is directed to the tip of the lead and consequently directly to the area of tissue fixation.

Another technique for removing the lead involves advancing a Cook catheter, i.e., a catheter commercially available from Cook Inc. of Bloomington Ind., with a cutting edged on the tip of the catheter, over and downward over the lead body toward the tip of the lead. Then, tissue can be cut away to free the side of the tip, and steady pulling techniques can be used to remove the lead. Still another way to remove a lead may involve advancing a laser catheter or an electro-cautery catheter over the lead, and then burning away tissue around or at the side of the lead tip. Steady pulling techniques can then be used to remove the lead. Still another way to remove the lead may involve direct surgical removal, often using temporary Cardio Pulmonary Bypass support as the heart is opened. In every case, when the tissue fixation structures provide very localized attachment to tissue, removal of the lead may not cause substantial tissue mutilation.

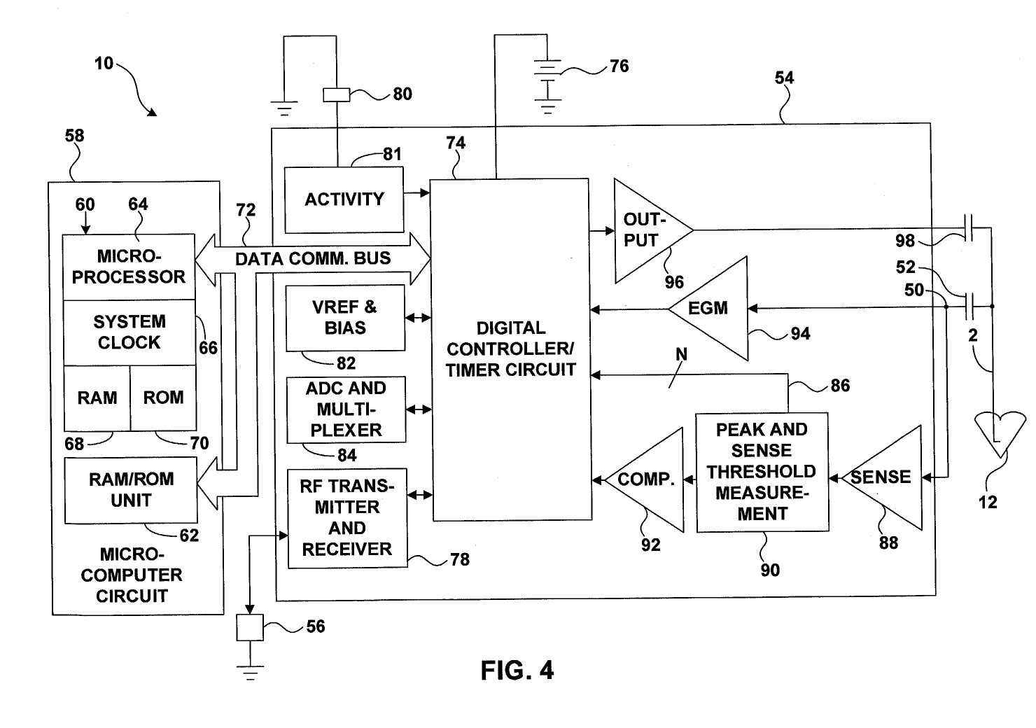

FIG. 4 is a block diagram illustrating the constituent components of pacemaker 10 that may make use of a lead 2 in accordance with the present invention. Pacemaker 10 may be a pacemaker having a microprocessor-based architecture. Pacemaker 10 is shown as including activity sensor or accelerometer 80, which is preferably a piezoceramic accelerometer bonded to a hybrid circuit located inside a housing (similar to control unit housing 4 illustrated in FIG. 1). Activity sensor 80 typically (although not necessarily) provides a sensor output to activity circuitry 81 that varies as a function of a measured parameter relating to a patient's metabolic requirements. Activity circuitry 81 may condition the signal, such as by filtering or analog-to-digital conversion, before forwarding the signal to digital controller 74. For the sake of convenience, pacemaker 10 in FIG. 4 is shown with lead 2 only connected thereto. However, it is understood that similar circuitry and connections not explicitly shown in FIG. 4 apply to any number of additional leads.

Pacemaker 10 in FIG. 4 is most preferably programmable by means of an external programming unit (not shown in the figures). One such programmer is the commercially available Medtronic Model 9790 programmer, which is microprocessor-based and provides a series of encoded signals to pacemaker 10, typically through a programming head which transmits or telemeters radio-frequency (RF) encoded signals to pacemaker 10. Such a telemetry system is described in U.S. Pat. No. 5,312,453 to Wyborny et al., hereby incorporated by reference herein in its entirety. The programming methodology disclosed in Wyborny et al.'s '453 patent is identified herein for illustrative purposes only. Any of a number of suitable programming and telemetry methodologies known in the art may be employed so long as the desired information is transmitted to and from the pacemaker. In this manner, pacemaker 10 can be programmed to perform one or more of the pacing and sensing techniques known in the art.

As shown in FIG. 4, lead 2 is coupled to node 50 in pacemaker 10 through input capacitor 52. Activity sensor or accelerometer 80 is most preferably attached to a hybrid circuit located inside hermetically sealed housing 42 of pacemaker 10. The output signal provided by activity sensor 80 is coupled to input/output circuit 54. Input/output circuit 54 contains analog circuits for interfacing with heart 12, activity sensor 80, antenna 56 and circuits for the application of stimulating pulses to heart 12. The rate of heart 12 is controlled by software-implemented algorithms stored within microcomputer circuit 58.

Microcomputer circuit 58 preferably comprises on-board circuit 60 and off-board circuit 62. Circuit 58 may correspond to a microcomputer circuit disclosed in U.S. Pat. No. 5,312,453 to Shelton et al., hereby incorporated by reference herein in its entirety. On-board circuit 60 preferably includes microprocessor 64, system clock circuit 66, on-board random access memory (RAM) 68 and read-only memory (ROM) 70. Off-board circuit 62 preferably comprises a RAM/ROM unit. On-board circuit 60 and off-board circuit 62 are each coupled by data communication bus 72 to digital controller/timer circuit 74. Microcomputer circuit 58 may comprise a custom integrated circuit device augmented by standard RAM/ROM components. In still other embodiments, the invention may be directed to an implantable medical device comprising one or more implantable leads that include electrodes and a control unit coupled to the electrodes via the leads. For example, the control unit may correspond to some or all the components of FIG. 4. The leads may include tissue fixation structures outlined in greater detail below.

The electrical components shown in FIG. 4 are powered by an appropriate implantable battery power source 76 in accordance with common practice in the art. For the sake of clarity, the coupling of battery power to the various components of pacemaker 10 is not shown in the Figures.

Antenna 56 is connected to input/output circuit 54 to permit uplink/downlink telemetry through RF transmitter and receiver telemetry unit 78. By way of example, telemetry unit 78 may correspond to that disclosed in U.S. Pat. No. 4,566,063 issued to Thompson et al., hereby incorporated by reference herein in its entirety, or to that disclosed in the above-referenced '453 patent to Wybomy et al. It is generally preferred that the selected programming and telemetry scheme permit the entry and storage of cardiac rate-response parameters. The specific embodiments of antenna 56, input/output circuit 54 and telemetry unit 78 presented herein are shown for illustrative purposes only, and are not intended to limit the scope of the present invention.

Continuing to refer to FIG. 4, VREF and Bias circuit 82 most preferably generates stable voltage reference and bias currents for analog circuits included in input/output circuit 54. Analog-to-digital converter (ADC) and multiplexer unit 84 digitizes analog signals and voltages to provide “real-time” telemetry intracardiac signals and battery end-of-life (EOL) replacement functions. Operating commands for controlling the timing of pacemaker 10 are coupled from microprocessor 64 via data bus 72 to digital controller/timer circuit 74, where digital timers and counters establish the overall escape interval of the pacemaker 10 as well as various refractory, blanking and other timing windows for controlling the operation of peripheral components disposed within input/output circuit 54.

Digital controller/timer circuit 74 is preferably coupled to sensing circuitry, including sense amplifier 88, peak sense and threshold measurement unit 90 and comparator/threshold detector 92. Circuit 74 is further preferably coupled to electrogram (EGM) amplifier 94 for receiving amplified and processed signals sensed by lead 2. Sense amplifier 88 amplifies sensed electrical cardiac signals and provides an amplified signal to peak sense and threshold measurement circuitry 90, which in turn provides an indication of peak sensed voltages and measured sense amplifier threshold voltages on multiple conductor signal path 86 to digital controller/timer circuit 74. An amplified sense amplifier signal is also provided to comparator/threshold detector 92. By way of example, sense amplifier 88 may correspond to that disclosed in U.S. Pat. No. 4,379,459 to Stein, hereby incorporated by reference herein in its entirety. Further, digital controller/timer circuit 74 can be programmed to execute various pacing techniques known in the art.

The electrogram signal provided by EGM amplifier 94 is employed when pacemaker 10 is being interrogated by an external programmer to transmit a representation of a cardiac analog electrogram. See, for example, U.S. Pat. No. 4,556,063 to Thompson et al., hereby incorporated by reference herein in its entirety. Output pulse generator 96 provides amplified pacing stimuli to patient's heart 12 through coupling capacitor 98 in response to a pacing trigger signal provided by digital controller/timer circuit 74 each time either (a) the escape interval times out, (b) an externally transmitted pacing command is received, or (c) in response to other stored commands as is well known in the pacing art. By way of example, output amplifier 96 may correspond generally to an output amplifier disclosed in U.S. Pat. No. 4,476,868 to Thompson, hereby incorporated by reference herein in its entirety.

The specific embodiments of sense amplifier 88, output pulse generator 96 and EGM amplifier 94 identified herein are presented for illustrative purposes only, and are not intended to be limiting in respect of the scope of the present invention. The specific embodiments of such circuits may not be critical to practicing embodiments of the present invention so long as they provide means for generating a stimulating pulse and are capable of providing signals indicative of natural or stimulated contractions of heart 12.

In some embodiments of the present invention, pacemaker 10 may operate in various non-rate-responsive modes. In other embodiments of the present invention, pacemaker 10 may operate in various rate-responsive modes. Some embodiments of the present invention may be capable of operating in both non-rate-responsive and rate-responsive modes. Moreover, in various embodiments of the present invention pacemaker 10 may be programmably configured to operate so that it varies the rate at which it delivers stimulating pulses to heart 12 in response to one or more selected sensor outputs being generated. Numerous pacemaker features and functions not explicitly mentioned herein may be incorporated into pacemaker 10 while remaining within the scope of the present invention

The present invention is not limited in scope to any particular number of leads or sensors, and is not limited to pacemakers comprising activity or pressure sensors only. In other words, at least some embodiments of the present invention may be applied equally well in the contexts of single-, dual-, triple- or quadruple-chamber pacemakers or other types of pacemakers. See, for example, U.S. Pat. No. 5,800,465 to Thompson et al., hereby incorporated by reference herein in its entirety, as are all U.S. Patents referenced therein. In each case, one or more of the leads may include tissue fixation structures to enhance fixation of the lead to cardiac tissue.

Pacemaker 10 may also be a pacemaker combined with a cardioverter and/or defibrillator. Various embodiments of the present invention may be practiced in conjunction with a pacemaker-cardioverter-defibrillator such as those disclosed in U.S. Pat. No. 5,545,186 to Olson et al., U.S. Pat. No. 5,354,316 to Keimel, U.S. Pat. No. 5,314,430 to Bardy, U.S. Pat. No. 5,131,388 to Pless, and U.S. Pat. No. 4,821,723 to Baker et al., all hereby incorporated by reference herein, each in its respective entirety.

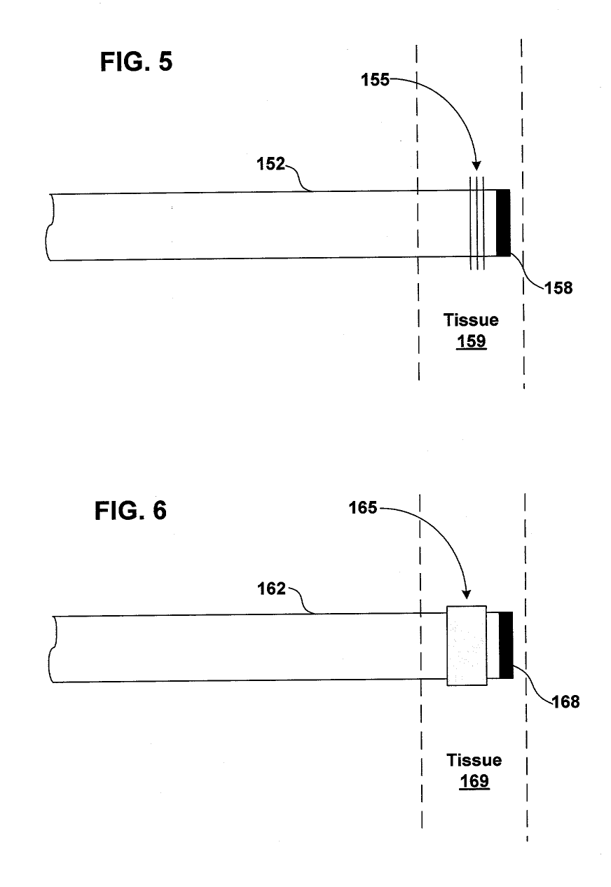

FIG. 5 is an enlarged cross-sectional side-view of one embodiment of the distal end of lead 152 in accordance with the invention. Lead 152 may correspond to lead 2 described above. In this example, lead 152 incorporates tissue fixation structures that promote fixation of the lead by tissue encapsulation and fibrous growth of tissue in the tissue site in which the lead is implanted. The tissue fixation structures may take the form of a set of ring structures 155 that extend radially from lead 152. For example, the ring-structures may be substantially similar to sealing gasket rings commonly provided on the proximal end of conventional leads for connection to control circuitry within a hermetically sealed housing of an implantable medical device.

In this case, however, ring structures 155 are positioned near the distal end of lead 152, a distance away from electrode 158. Ring structures 155 provide a larger surface area for tissue 159 to grow around, and a platform for attachment of the tissue. Thus, ring structures 155 may allow electrode 158 to be securely fixed with respect to a desired location within a patient. Also, lead 152 may be removed from the tissue 159 without causing substantial tissue mutilation because the fixation structures provide very localized attachment to tissue. In contrast, conventional hook structures or other aggressive fixation structures may cause substantial tissue mutilation upon removal by tearing larger portions of the tissue. In accordance with the invention, tissue mutilation upon removal of the lead may be very localized around the relatively small area associated with the fibrous growth structures. In other words, the tissue fixation structures may engage tissue in a very small contact area.

Ring structures can be formed by injection molding material against the insulating outer sleeve of the lead or by injection molding the rings as a separate part or component. In the later case, the rings can be applied to the lead by an adhesive or the like. As an example, the rings may be 0.5-2 mm wider than a diameter of the lead body in order to ensure that they can be removed without causing substantial tissue mutilation. The rings need only to be somewhat oversized relative lead body in order to ensure that tissue formation around the rings will secure the lead.

The formation of tissue is typically quite rapid, and should start almost immediately after the initial trauma caused by lead placement. Once the initial inflammation occurs, the healing process is immediately activated. Within a day or so, fixation starts to be become realized as the body start to cope with the foreign substance. The process then continues for weeks to follow, while the stimulation threshold gradually increases due to the formation of the fibrous tissue around the electrode tip.

Due to the increased resistance of the structures against the tissue, the lead should not slip as easily from its position than a standard lead without structures. Furthermore, the increased inward pressure of the surrounding tissues around the lead body, with it's fixation structures, will firmly grasp the lead around the structures. The presence of the fixation structures themselves will also promote inflammation, so tissue will also quickly start to grow around these structures as well.

FIG. 6 is another close-up cross-sectional side-view of one embodiment of the distal end of a lead 162 in accordance with the invention. Lead 162 may correspond to lead 2 described above. In this example, lead 162 incorporates tissue fixation structures in the form of a porous material 165 formed on lead 162. For example, porous material 165 can be formed on lead near the distal end of lead 162, a distance away from electrode 168. Porous material 165 may provide an improved growth surface for tissue 169 to grow and attach to lead 162. Thus, porous material 165 may allow electrode 168 to be securely fixed with respect to a desired location within a patient. Also, lead 162 may be removed from the tissue 169 without causing substantial tissue mutilation. An abrasive material may also be used in place of, or in addition to porous material 165.

By way of example, the porous materials used may be made of sintered (heated) platinum particles, or of pyrolytic carbon. The pores may be very tiny, ranging from 0.0001-0.01 mm in diameter, and up to about 0.1 mm deep, although the invention is not necessarily limited in that respect. The pores may have regular or irregular distribution. In other examples, rough surfaces may be provided via sintered Platinum Particles, or a saw tooth, or a beaded design as outlined in greater detail below.

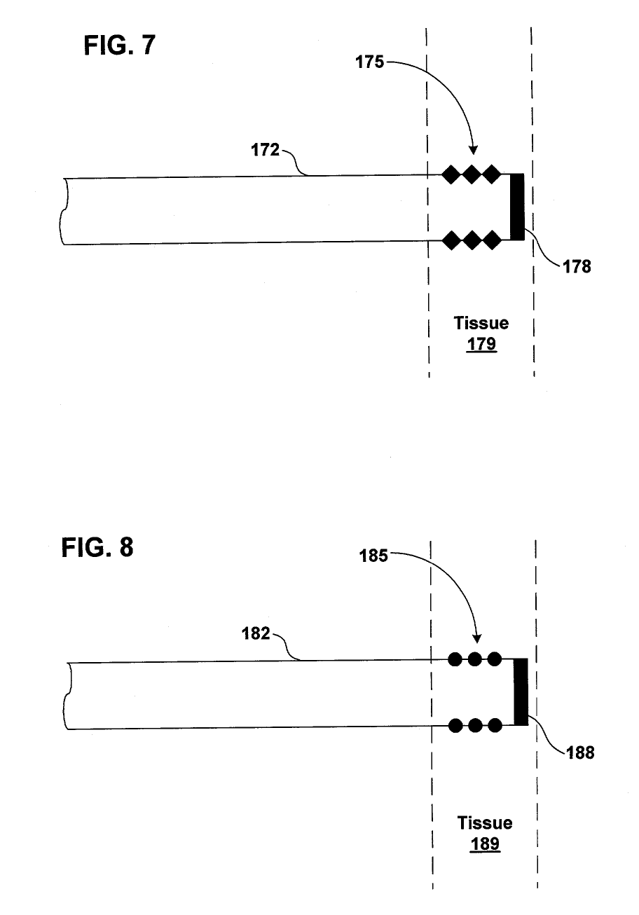

FIG. 7 is a close-up cross-sectional side-view of one embodiment of the distal end of a lead 172 in accordance with the invention. Lead 172 may correspond to lead 2 described above. In this example, lead 172 incorporates tissue fixation structures in the form of a saw-tooth surface 175 formed on lead 172. For example, saw-tooth surface 175 can be formed on lead 172 near the distal end of lead 172, a distance away from electrode 178. The saw-tooth surface 175 may provide increased surface area for tissue 179 to grow and attach thereto. Thus, saw-tooth surface 175 may allow electrode 178 to be securely fixed with respect to a desired location within a patient. Again, lead 172 may be removed from the tissue 179 without causing substantial tissue mutilation.

The saw tooth structures can be formed in the mold from which a polymer lead is formed, or by mechanical machining, e.g., of metal components. For example, silicone can be injected in the mold and vulcanized, or polyurethane may be injection molded to form the desired surface structures. In both techniques, the polymer can be applied to the insulating outer sleeve of the lead body or by making separate components which later are glued or affixed to the lead body. The depth and width of each saw tooth may be on the order of 0.1 to 0.5 mm in order to ensure that adequate fixation can be achieved, and also to ensure that removal without substantial tissue mutilation can be achieved. The number of teeth may be between 5 and 50 although the invention is not necessarily limited in that respect.

FIG. 8 is a close-up cross-sectional side-view of one embodiment of the distal end of a lead 182 in accordance with the invention. Lead 182 may correspond to lead 2 described above. In this example, lead 182 incorporates tissue fixation structures in the form of a beaded surface 185 formed on lead 182. For example, beaded surface 185 can be formed on lead near the distal end of lead 182, a distance away from electrode 188. The beaded surface 185 may provide increased surface area for tissue 189 to grow and attach thereto. Thus, beaded surface 185 may allow electrode 188 to be securely fixed with respect to a desired location within a patient. Again, lead 182 may be removed from the tissue 189 without causing substantial tissue mutilation.

The beaded surface may be formed of platinum beads welded or sintered to the surface of a metal base. Alternatively the beads may be made of silicone or polyurethane, or some other polymer material that can be vulcanized to the lead body. Bead diameters may range from 0.01 to 0.1 mm in order to ensure that adequate fixation can be achieved and that removal without substantial tissue mutilation can also be achieved. The beads may be suspended in an emulsion that is applied to a solid surface. The emulsion solidifies and the assembly can be placed in an oven. The temperature within the oven can be increased to approximately the melting temperature of the metal. The emulsion that kept the beads in place will evaporate while the beads melt together where they make contact. The evaporated suspension material may form the space of the later pores. The area covered by the beaded coating may spread over part of all of the circumference of the lead and may have an axial dimension on the order of approximately 1 to 5 mm, although the invention is not necessarily limited in that respect.

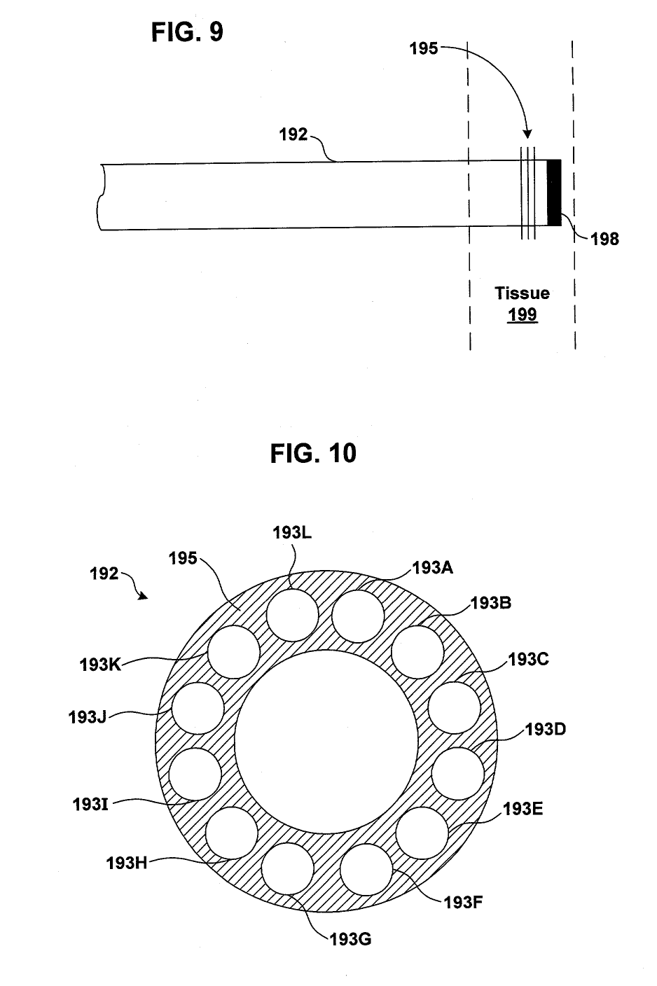

FIG. 9 is a close-up cross-sectional side-view of one embodiment of the distal end of a lead 192 in accordance with the invention. Lead 192 may correspond to lead 2 described above. In this example, lead 192 incorporates tissue fixation structures in the form of a set of ring structures 195 similar to ring structures 155 (FIG. 5). For example, the ring-structures may be similar to sealing gasket rings commonly provided on the proximal end of lead 192 that connects to control circuitry of an implantable medical device, or may comprise a different material. In either case, ring structures 192 can be formed to include fibrous growth holes formed in the rings as further illustrated in FIG. 10. Ring structures 192 may extend radially from lead 192.

FIG. 10 is a close-up front-view of lead 192 illustrating fibrous growth holes 193A-193L (collectively fibrous growth holes 193) formed in one or more of the ring structures 195. Fibrous growth holes 193 can significantly enhance the fixation effect associated with fibrous tissue growth around ring structures 195. In particular, tissue may form and connect between the fibrous growth holes 193 eventually providing a web of tissue that is connected on either side of the various ring structures 195 and connected through the fibrous growth holes 193. In this manner, tissue growth between the fibrous growth holes 193 can significantly improve fixation of lead 192 within tissue 199. In other words, tissue growth through the fibrous growth holes 193 can serve to anchor lead 192 within tissue 199 thereby securely fixing electrode 198 with respect to a desired location within a patient. Still, tissue mutilation upon removal of the lead may be very localized around the relatively small area associated with the ring structures 195 and fibrous growth holes 193. Thus, lead 192 may be removed from the tissue 199 without causing substantial tissue mutilation.

The growth holes may be formed as part of the injection mold used to create the ring structures. By way of example, if a ring extends by 0.5-2 mm, the holes can be adjusted to the width of the rings. The diameter of the holes may be on the order of approximately 0.1-0.55 mm, although the invention is not necessarily limited in that respect. The holes may extend all the way through the rings in order to allow tissue to grow in from both sides and form a contiguous tissue structure that has a structural strength. The force needed to fracture the tissue in the holes may be directly related to the number of holes and the sizes of the holes. Thus, more holes may provide more fixation strength.

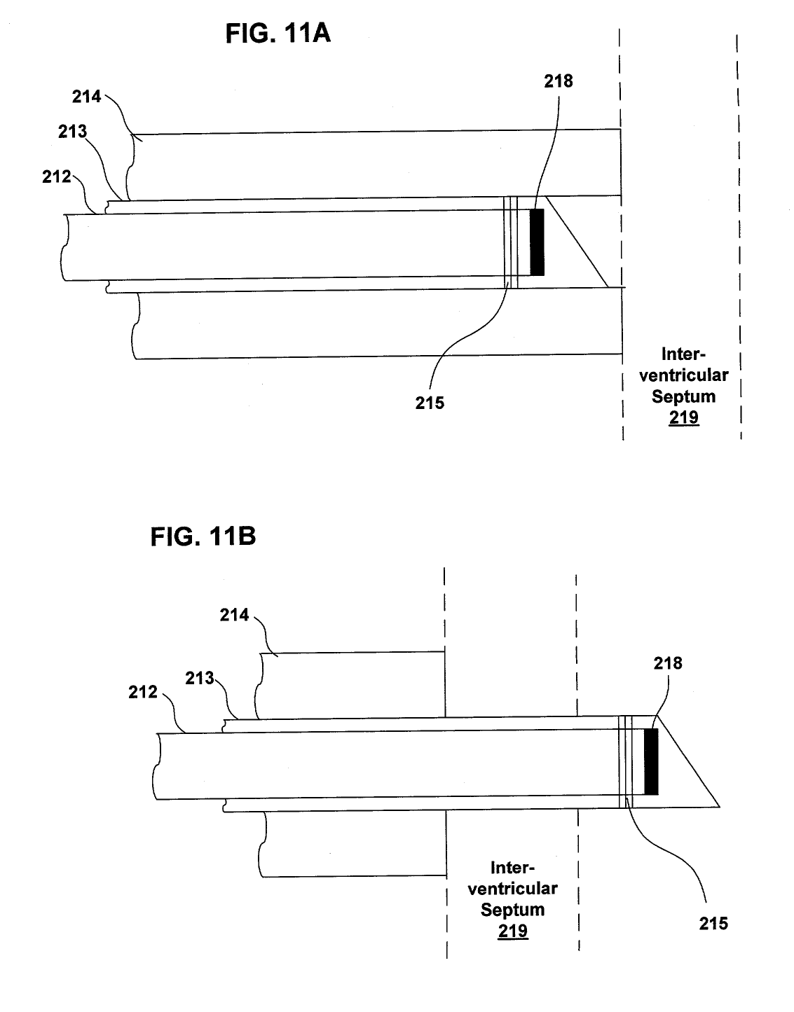

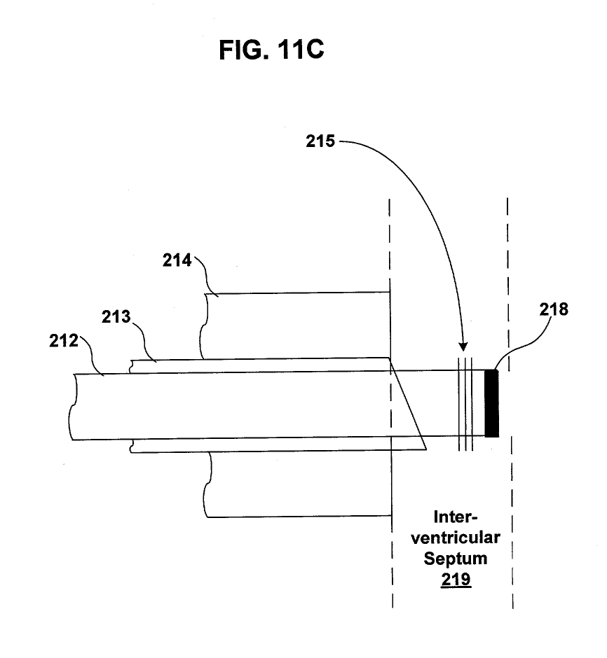

FIGS. 11A-11C are close-up cross-sectional side-views collectively illustrating the insertion and placement of a lead within tissue. For exemplary purposes, FIGS. 11A-11C are described in the context of insertion and placement of the lead within the interventricular septum. However, the same or similar techniques may be used to insert and place a lead with respect to other cellular tissue. As shown in FIG. 11A, lead 212 is originally surrounded by puncture needle 213. Lead 212 may correspond to lead 2 described above. A guiding catheter 214 can be used to guide the lead 212 and puncture needle 213 to the desired location within the patient. In this case, guiding catheter 214 guides the lead 212 and puncture needle 213 to the interventricular wall.

As shown in FIG. 11B, once guiding catheter 214 has guided lead 212 and puncture needle 213 to the interventricular wall, puncture needle is used to puncture the interventricular septum 219. Then, as shown in FIG. 11C, the puncture needle can be retracted and lead 212 can be positioned within the interventricular septum 219 such that electrode 218 is in close proximity to the left ventricle. Electrode 218 can then be used for sensing and/or stimulating the left ventricle as outlined above.

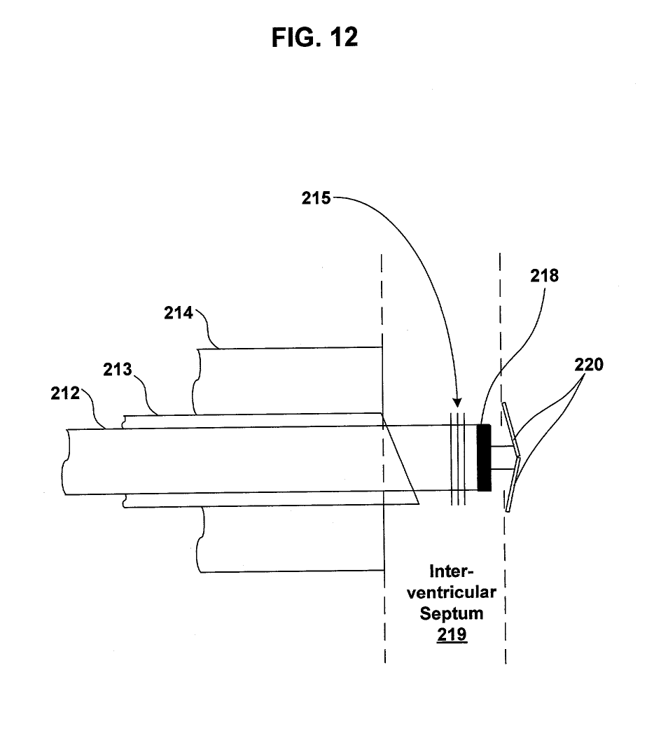

Over time, tissue growth within the punctured hole in the interventricular septum will anchor lead 212 within interventricular septum, and this anchoring effect is improved because lead 212 includes one or more of the tissue fixation structures 215, such as one or more of those outlined above. Tissue typically starts to form immediately after the start of inflammation process. Complete fibrous growth may take a few days or a few weeks depending on the patient. Guiding catheter 214 may also abut the wall of interventricular septum 219 to restrict forward motion of lead 212 with respect to interventricular septum 219. Optionally, lead 212 may also include distal tines 220 as illustrated in FIG. 12 to further restrict backward motion of lead 212 with respect to interventricular septum 219.

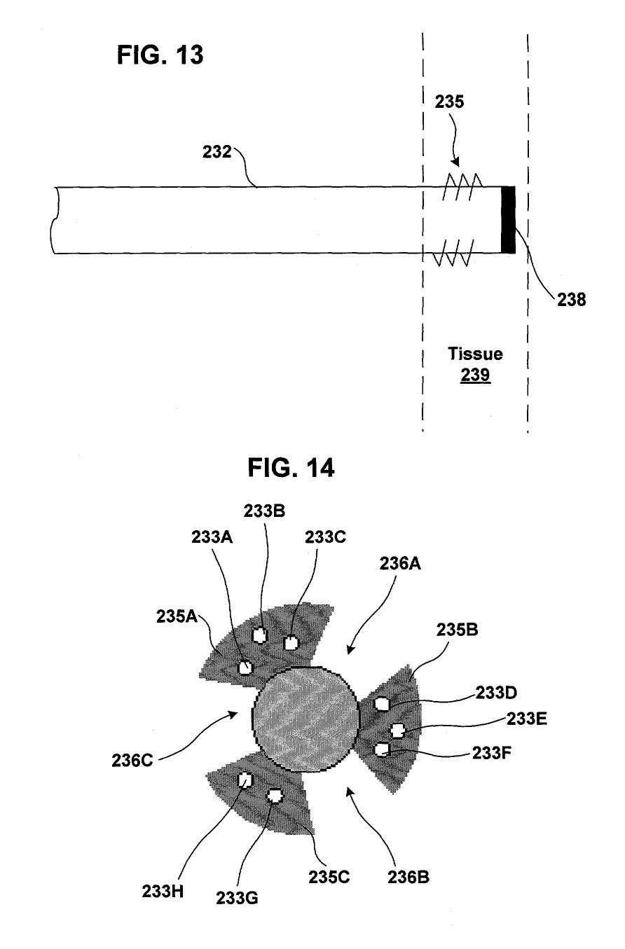

FIG. 13 is another close-up cross-sectional side-view of one embodiment of the distal end of a lead 232 in accordance with the invention. Lead 232 may correspond to lead 2 described above. In this example, lead 232 incorporates tissue fixation structures in the form of non-continuous spiral ring structures 235. The non-continuous spiral ring structures 235 may or may not be formed with fibrous growth holes to enhance fixation to tissue within tissue 239. In particular, non-continuous spiral ring structures 235 comprise a segmented threading that can be used to screw lead 232 into tissue 239 and thereby position electrode 238 with respect to a desired location. The non-continuous nature of non-continuous spiral ring structures 235 can enhance fibrous tissue growth around and between the non-continuous spiral ring structures 235 to improve fixation of lead 232 within tissue 239. Fibrous growth holes formed in the non-continuous spiral ring structures 235 may also improve fixation.

The spiral rings may be made from metal or rigid polymer material. The size and depths of the rings may be similar to depth of previously described continuous rings, therefore extending approximately 0.5-2.0 mm outward from lead body. Thus, the diameter of the non-continuous spiral rings may be approximately 2*(0.5-2 mm) larger than lead body diameter. By way of example, the pitch of the spiral rings may range from about 1 to 3 mm, although the invention is not necessarily limited in that respect.

The non-continuous spiral rings can be formed in an injection mold and may be made from a non-corrosive steel alloy. The holes in the spirals may be formed by corresponding cylindrical pins that are placed in the mold prior to injection, and then removed from the mold prior to the opening of the mold, or during the opening of the mold.

FIG. 14 is a close-up front-view of non-continuous spiral ring structures 235A-235C (collectively non-continuous spiral ring structures 235) formed with fibrous growth holes 233A-233H (collectively fibrous growth holes 233). Non-continuous spiral ring structures 235 are spiraled with respect to one another, and intermittent open spaces 236A-236C (collectively gaps 236) separate respective non-continuous spiral ring structures 235. The non-continuous spiral ring structures 235 as well as fibrous growth holes 233 improve fixation of lead 232 to tissue within tissue 239. Still, lead 232 may be removed from tissue 239 without causing substantial tissue mutilation simply by screwing lead 232 out of tissue 239. In that case, tissue damage would be similar to the damage incurred when the lead is inserted into the tissue 239.

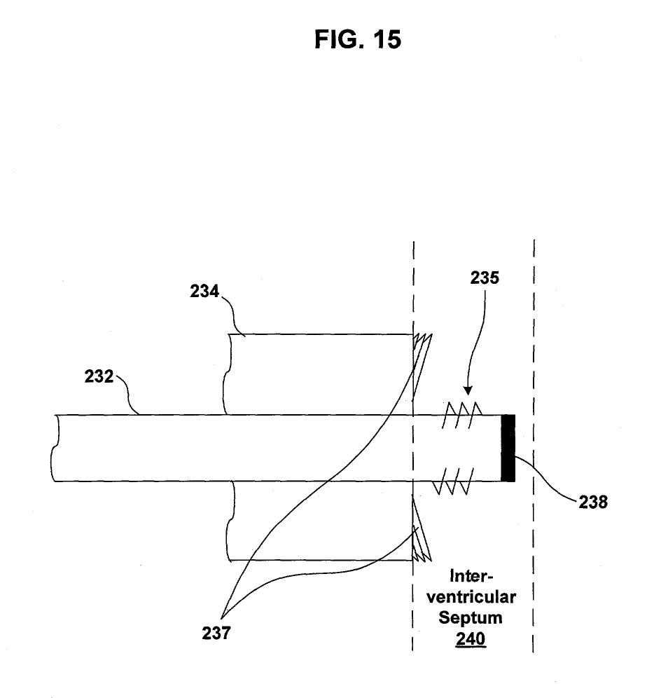

FIG. 15 is a close-up cross-sectional side-view illustrating the placement of the lead illustrated in FIGS. 13 and 14 within tissue. For exemplary purposes, FIG. 15 is described in the context of insertion and placement of the lead within the interventricular septum. However, the same or similar techniques may be used to insert and place a lead with respect to other cellular tissue. Lead 232 is originally surrounded by guiding catheter 234, which can be used to guide the lead 232 to the desired location within the patient. In this case, guiding catheter 234 guides the lead 232 to the interventricular wall. Guiding catheter 234 has anchoring features 237 such as screw-like spiraled grooves that can be used to fix the catheter 234 to the interventricular wall. Then, lead 232 can be screwed into the interventricular septum 240 to a desired location with respect to the left ventricle. Again, non-continuous spiral ring structures 235 as well as fibrous growth holes 233 improve fixation of lead 232 to tissue within interventricular septum 240.

The preceding specific embodiments are illustrative of the practice of the invention. It is to be understood, however, that in light of this disclosure, other embodiments will become apparent to those skilled in the art. For example, some embodiments may be practiced in an external (non-implantable) or a partially external medical devices. Also, the invention may also be used for implantable devices that sense and stimulate neurological or muscular tissue. If used with a cardiac pacemaker, the invention may improve lead fixation to cardiac tissue such as the inter-ventricular septum, the intra-ventricular wall, the coronary sinus, cardiac veins, or any other cardiac tissue. Accordingly, these and other embodiments are within the scope of the following claims.

In the claims, means-plus-function clauses are intended to cover the structures described herein as performing the recited faction and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw are equivalent structures.

1. An implantable medical lead comprising:
a lead including a proximal end, a distal end, and an electrode; and
tissue fixation structures on the lead a distance from the electrode, wherein the tissue fixation structures promote fibrous tissue growth with respect to the lead to enhance fixation of the lead to tissue.

2. The implantable medical lead of claim 1, wherein the lead includes an electrode is positioned at the distal end, and the tissue fixation structures are on the lead in proximity to the distal end a distance from the electrode.

3. The lead of claim 1, wherein the tissue fixation structures are formed to facilitate removable fixation of the lead to tissue without causing substantial tissue mutilation of the tissue upon removal.

4. The lead of claim 1, wherein the tissue fixation structures comprise rings that extend radially outward from the lead.

5. The lead of claim 4, wherein the rings define holes that promote fixation of the lead via fibrous growth of tissue with respect to the holes.

6. The lead of claim 1, wherein the tissue fixation structures comprise an abrasive material on the lead.

7. The lead of claim 1, wherein the tissue fixation structures comprise a saw-tooth structure on the lead.

8. The lead of claim 1, wherein the tissue fixation structures comprise a beaded coating on the lead.

9. The lead of claim 1, wherein the tissue fixation structures comprise non-continuous spiral rings separated by intermittent open spaces.

10. The lead of claim 9, wherein the non-continuous spiral rings define fibrous growth holes.

11. The lead of claim 1, wherein the tissue fixation structures define fibrous growth holes.

12. The lead of claim 1, further comprising distal tines disposed at the distal end of the lead to restrict backwards motion of the lead.

13. An implantable medical device comprising:
a medical lead including a proximal end, a distal end, and an electrode, and tissue fixation structures defined on the lead a distance from the electrode, wherein the tissue fixation structures increase fibrous tissue growth with respect to the lead to enhance fixation of the lead to tissue and thereby secure the electrode in an implanted position; and
a puncture needle that substantially encapsulates the lead.

14. The implantable medical device of claim 13, further comprising a guiding catheter that substantially encapsulates the puncture needle.

15. The device of claim 13, wherein the electrode is positioned at the distal end, and the tissue fixation structures are defined on the lead in proximity to the distal end a distance from the electrode.

16. The device of claim 13, wherein the tissue fixation structures are defined to facilitate removable fixation to tissue without causing substantial tissue mutilation upon removal.

17. The device of claim 13, wherein the tissue fixation structures comprise a set of rings that extend from the lead.

18. The device of claim 17, wherein the rings include fibrous growth holes defined in the rings.

19. The device of claim 13, wherein the tissue fixation structures comprise an abrasive material on the lead.

20. The device of claim 13, wherein the tissue fixation structures comprise a saw-tooth structure on the lead.

21. The device of claim 13, wherein the tissue fixation structures comprise a beaded coating on the lead.

22. The device of claim 13, wherein the tissue fixation structures comprise non-continuous spiral rings separated by intermittent open spaces.

23. The device of claim 13, wherein the non-continuous spiral rings include fibrous growth holes in the non-continuous spiral rings.

24. The device of claim 13, wherein the tissue fixation structures include fibrous growth holes.

25. The device of claim 13, further comprising distal tines disposed at the distal end of the lead to restrict backwards motion of the lead.

26. An implantable medical device comprising:
a medical lead including a proximal end, a distal end, and an electrode, and tissue fixation structures on the lead a distance from the electrode, wherein the tissue fixation structures exploit fibrous tissue growth with respect to the lead to enhance fixation of the lead to tissue; and
a control unit coupled to the proximal end.

27. The device of claim 26, wherein the control unit controls stimulation pulses delivered by the electrode.

28. The device of claim 26, wherein the control unit processes signals detected by the electrode.

29. The device of claim 26, wherein the device is a cardiac pacemaker

30. The device of claim 26, wherein the device is a neurological stimulation device.

31. The device of claim 26, wherein the device is a muscular stimulation device.

32. The device of claim 26, wherein the electrode is positioned at the distal end, and the tissue fixation structures are on the lead in proximity to the distal end a distance from the electrode.

33. The device of claim 26, wherein the tissue fixation structures are formed to facilitate removable fixation to tissue without causing substantial tissue mutilation upon removal.

34. The device of claim 26, wherein the tissue fixation structures comprise a set of rings that extend from the lead.

35. The device of claim 34, wherein the rings include fibrous growth holes in the rings.

36. The device of claim 26, wherein the tissue fixation structures comprise an abrasive material on the lead.

37. The device of claim 26, wherein the tissue fixation structures comprise a saw-tooth structure on the lead.

38. The device of claim 26, wherein the tissue fixation structures comprise a beaded coating on the lead.

39. The device of claim 26, wherein the tissue fixation structures comprise non-continuous spiral rings separated by intermittent open spaces.

40. The device of claim 26, wherein the non-continuous spiral rings include fibrous growth holes in the non-continuous spiral rings.

41. The device of claim 26, wherein the tissue fixation structures include fibrous growth holes.

42. The device of claim 26, further comprising distal tines disposed at the distal end of the lead to restrict backwards motion of the lead.

43. An implantable medical lead comprising:
a lead having a proximal end, a distal end, and an electrode; and
means for tissue fixation on the lead a distance from the electrode, wherein the
means exploits fibrous tissue growth with respect to the lead to enhance fixation of the lead to tissue and thereby secure the electrode in an implanted position.

44. The implantable medical lead of claim 43, further comprising, means for improving fibrous tissue growth with respect to the means for tissue fixation.

45. A method comprising:
puncturing a hole in an interventricular septum with a puncture needle that encapsulates a medical lead including tissue fixation structures on a lead a distance from an electrode on the distal end of the lead;
retracting the puncture needle from the punctured hole; and
retracting the medical lead such that the tissue fixation structures are positioned within the interventricular septum.

46. The method of claim 20, further comprising retracting the medical lead such that the electrode is positioned within the interventricular septum in close proximity to a ventricle.

47. The method of claim 45, wherein the tissue fixation structures include fibrous growth holes.

48. The method of claim 45, further comprising guiding the puncture needle and the lead to the interventricular septum prior to puncturing the interventricular septum.

49. The method of claim 45, further comprising removing the lead from the interventricular septum without causing substantial tissue mutilation to the interventricular septum.

50. An implantable medical lead comprising:
a lead including a proximal end, a distal end, and an electrode; and
tissue fixation structures that extend a distance radially from the lead, wherein the tissue fixation structures exploit fibrous tissue growth with respect to the lead to enhance fixation of the lead to tissue.

51. The implantable lead of claim 50, wherein the tissue fixation structures comprise ring structures that extend radially from the lead.

52. The implantable lead of claim 50, wherein the tissue fixation structures comprise non-continuous spiral rings separated by intermittent open spaces.

53. The implantable lead of claim 50, wherein the tissue fixation structures include fibrous growth holes.

54. An implantable medical device comprising:
a medical lead including a lead having a proximal end, a distal end, and an electrode, and tissue fixation structures that extend a distance radially from the lead, wherein the tissue fixation structures exploit fibrous tissue growth with respect to the lead to enhance fixation of the lead to tissue; and
a control unit coupled to the proximal end.

55. The device of claim 54, wherein the tissue fixation structures comprise ring structures that extend radially from the lead.

56. The device of claim 54, wherein the tissue fixation structures comprise non-continuous spiral rings separated by intermittent open spaces.

57. The device of claim 54, wherein the tissue fixation structures include fibrous growth holes.
